(19) United States
(12) Patent Application Publication
(10) Pub. No.: US 2021/0349108 A1
(43) Pub. Date: Nov. 11, 2021

(54) IMMUNOASSAY FOR MEASURING C-PEPTIDE

(71) Applicant: Roche Diabetes Care GmbH, Indianapolis, IN (US)

(72) Inventors: Arnulf Staib, Heppenheim (DE); Alexander Licht, Weinheim (DE); Daniel Kammerer, Heidelberg (DE)

(73) Assignee: Roche Diabetes Care GmbH, Indianapolis, IN (US)

(21) Appl. No.: 17/284,586

(22) PCT Filed: Oct. 15, 2019
(86) PCT No.: PCT/EP2019/077971

§ 371 Date: Apr. 12, 2021

(30) Foreign Application Priority Data
Oct. 15, 2018 (EP) 18200435.8

(51) Int. Cl. G01N 33/74; C07K 14/62; G01N 33/543

(52) U.S. Cl. CPC G01N 33/74; C07K 14/62; G01N 2800/042; G01N 2333/62; G01N 2470/12; G01N 33/54387

(57) ABSTRACT

The present invention relates to a method for detecting and in particular for measuring C-peptide. Specifically, the present invention relates to a displacement assay for quantitative determination of C-peptide in a sample.

The present invention relates to a method for detecting and in particular for measuring C-peptide. Specifically, the present invention relates to a displacement assay for quantitative determination of C-peptide in a sample.

Insulin is produced within the human body from proinsulin. In the pancreas proinsulin is cleaved by proteases whereby insulin and C-peptide are produced in equimolar amounts. However, due to a longer halftime the concentration of C-peptide is about five times higher than the concentration of insulin in the blood circuit. Therefore, C-peptide serves as an target analyte in assays directed to determination of insulin and conditions related thereto, such as Type 1 or Type 2 diabetes.

C-peptide and insulin can e.g. be measured using sandwich immunoassays as described in A. G. Jones and A. T. Hattersley, Diabet Med. 2013, 30(7): 803-817. Also in U.S. Pat. No. 6,534,281 and in U.S. Pat. No. 6,929,924 C-peptide immunoassays are described. However sandwich immunoassays for determination of human C-peptide require a complex set-up including multiple handling and washing steps. Often a large sample volume is necessary making these assays unsuitable for point-of-care diagnostics.

WO 95/02703 A1 describes lyophilized ligand-receptor complexes for assays and sensors, which on rehydration can be used for detecting analytes in samples in a variety of displacement assays. According to WO 95/02703 A1, in particular a fluorescently labeled benzoylecgonine is used as ligand in an assay to detect the analyte benzoylecgonine.

WO 2005/118638 A1 describes degradation-resistant analogues of pro-insulin C-peptide. U.S. 2016/0355564 A1 relates to a method to detect autoantibody reactivity for deaminated insulin autoantigen in diabetes.

An objective of the present invention was therefore to provide an improved method for measuring C-peptide.

This objective is solved according to the invention by a method for detecting C-peptide comprising
i) providing an antibody-ligand-complex, wherein the antibody is an antibody directed against C-peptide (i.e. an anti-C-peptide antibody) and
wherein the ligand has a lower affinity to the anti-C-peptide antibody than wild type C-peptide and
ii) contacting the antibody-ligand-complex with a sample to be analyzed and
iii) displacement of the ligand from the antibody-ligand-complex by C-peptide analyte, thereby determining the presence or concentration of C-peptide analyte within the sample.

Preferably, the inventive method is for detecting human C-peptide and in particular for detecting human wild type C-peptide. Human wild type C-peptide herein also termed wild type C-peptide or WT-C-peptide, is a peptide consisting of 31 amino acids having the sequence

SEQ ID NO: 1
N-EAEDLQVGQVELGGGPGAGSLQPLALEGSLQ-C.
(= WT-C-peptide)

The 31 amino acids of human wild type C-peptide constitute amino acids 33-63 of human proinsulin.

According to the present invention in particular human wild type C-peptide having the sequence SEQ ID NO: 1 is the target analyte.

The inventive method for detecting C-peptide is in particular an immunoassay for measuring C-peptide using a ligand displacement method. In such a displacement assay only one antibody is required for detection of the target analyte C-peptide.

According to the invention an antibody-ligand-complex is provided. Such antibody-ligand-complex may be in soluble form, but is preferably provided immobilized to a solid phase. To form an antibody-ligand-complex an antibody is contacted with a ligand.

The antibody employed is directed against C-peptide. Such an antibody directed against C-peptide is also called anti-C-peptide antibody herein. Preferably, an antibody is employed which has a high specificity and affinity towards wild type C-peptide. In particular the anti-C-peptide antibody has an affinity K_{aff} of ≥10⁷ [1/M], in particular ≥10⁸ [1/M] or ≥10⁹ [1/M] against C-peptide, in particular against human wild type C-peptide having the sequence SEQ ID NO: 1. Such antibodies are known in the art.

According to the invention polyclonal or monoclonal antibodies can be used. Monoclonal antibodies are preferred.

Examples of suitable anti-C-peptide antibodies are anti-human C-peptide monoclonal antibody 9101 (Medix Biochemica), anti-human C-peptide monoclonal antibody 9103 (Medix Biochemica), anti-human C-peptide monoclonal antibody CPT-3-F11 (Dako) and preferably anti-human C-peptide monoclonal antibody PEP-001 (Dako) or anti-human C-peptide monoclonal antibody 7E10 (Thermo Fisher Scientific). Thus, commercially available anti-human C-peptide antibodies and in particular anti-human C-peptide monoclonal antibodies can be employed in the inventive method. Most preferably anti-human C-peptide monoclonal antibody PEP-001 is used. The central binding motif for the PEP001 antibody is LQP.

An essential advantage of the inventive method in the format of an displacement assay is that only one single antibody is required. This is in contrast to immunoassays in the sandwich format, in which as at least two antibodies are necessary.

The antibody-ligand-complex provided in the inventive method further comprises a ligand. The ligand, which is later in the method to be displaced by the C-peptide analyte, in particular by human wild type C-peptide is selected to have a lower affinity to the anti-C-peptide antibody than wild type C-peptide. In particular the ligand has an affinity K_{aff} of less than 90%, preferably less than 50%, more preferably less than 10% of K_{aff} of the wild type C-peptide to the anti-C-peptide antibody. Thus, according to the invention K_{aff ligand} K_{aff WT-C-peptide}, in particular K_{aff ligand}<0.9×K_{aff WT-C-peptide}, preferably K_{aff ligand}<0.5×K_{aff WT-C-peptide}, and more preferably K_{aff} ligand<0.1×K_{aff WT-C-peptide}. Preferred ligands are C-peptide variants having at least one amino acid substitution and/or deletion as described herein. In one embodiment, the ligand is an optionally truncated variant of the peptide of SEQ ID NO:1, wherein at least one and up to 5 amino acids are substituted and preferably, wherein one or two amino acids are substituted, in particular one. In a further embodiment the ligand is a truncated variant of the peptide of SEQ ID NO:1, with no amino acid substitution.

According to the invention the antibody-ligand-complex is contacted with a sample. Suitable samples are e.g. urine, blood, plasma or serum from a subject. Preferably the antibody-ligand-complex is immobilized on a solid phase and then contacted with a liquid sample to be analyzed. The antibody-ligand-complex can in particular be immobilized on a solid phase via the antibody or via the ligand.

C-peptide analyte and in particular wild type C-peptide being present in the sample then displaces the ligand from the antibody-ligand-complex due to the higher affinity of C-peptide analyte and in particular wild type C-peptide to the antibody compared to the affinity of the ligand to the antibody. In one preferred embodiment the displaced ligand is then present in free form within the sample. In a further preferred embodiment the displaced ligand remains immobilized on a solid phase and the released antibody bound to wild type C-peptide is present within the sample.

According to the inventive method the presence of C-peptide analyte and in particular wild type C-peptide within the sample can be detected and preferably quantitatively determined. This detection or quantitative determination, respectively, can be performed in different ways, for example by measurement of the released ligands, via markers attached to the ligand or label-free, e.g. by weight measurement, or by impedance measurement.

The inventive detection of C-peptide using a displacement assay provides several advantages. A displacement assay can in particular be conducted in a point-of-care setting, under use of test strips without great effort and without need for laboratory equipment. Because C-peptide is a small linear peptide, it is susceptible to degradation in samples like urine, serum or blood. The analysis should be done in a short time window after collecting the sample and cooling and fast sample processing is required. Using a displacement assay, there is no need to transport the sample to an analytical laboratory. Without the need of many handling steps or washing steps a displacement assay provides a fast result within a short time of some seconds up to few minutes Additionally, the assay format enables the performance of the method using only a small sample volume in the microliter range. In sum, the inventive method allows for an easy test performance and at the same time provides high sensitivity.

In known displacement assays (cf. for example Hinds et al, Clin. Chem., 1984, 30, 7, 1174-1178; U.S. Pat. Nos. 4,434,236; 5,527,686 or 5,573,921) antibodies were used having a low affinity or cross reactivity. Only by using those antibodies having low affinity or cross reactivity displacement of a labeled ligand from its binding to an antibody by a targeted analyte was possible. When using an antibody having high affinity to the target analyte and to a labeled ligand to be displaced only a small part of the antibody bound labeled molecules are displaced resulting in low sensitivity of this test format. Therefore, displacement assays are not very common in commercial applications and are mainly used for low molecular weight analytes.

According to the invention an antibody with high affinity to the target analyte C-peptide can be used, which is made possible by employing a ligand which has only a low affinity to the anti-C-peptide antibody. It was found that by modification of the targeted analyte C-peptide a ligand can be provided which has a lower affinity to the antibody than the wild type C-peptide. Ligands suitable in the inventive method are in particular provided by modification of wild type C-peptide by one or more amino acid substitutions and/or deletions. According to the invention thereby ligands are provided which are bound by the anti-C-peptide antibody used, at the same time however can be displaced from the binding to the antibody by C-peptide analyte and in particular wild type C-peptide.

In a preferred embodiment the ligand is a C-peptide variant. A C-peptide variant is derived from the wild type C-peptide as shown in SEQ ID NO: 1, but has at least one amino acid insertion, substitution and/or deletion, in particular at least one substitution and/or deletion. A C-peptide variant therefore differs from wild type C-peptide in a change in the sequence. Correspondingly, while the ligand of the present invention and, in particular a C-peptide variant as ligand according to the present invention may carry a label, a wild type C-peptide carrying a label is not within the definition of a C-peptide variant. A C-peptide variant preferably shows at least one and up to 10, preferably at least one and up to 5, more preferably at least one and up to 2 and most preferably exactly one substitution over the wild type C-peptide. Additionally or alternatively, the C-peptide variant shows 1 to 23, preferably 5 to 20 and more preferably 10 to 16 deletions compared to wild type C-peptide. Correspondingly, a C-peptide variant comprises preferably up to 30 amino acids in accordance with the wild type C-peptide sequence, preferably up to 25 amino acids and more preferably up to 20 amino acids and at least 7 amino acids, more preferably at least 9 amino acids and even more preferably at least 10 amino acids in accordance with the wild type C-peptide sequence. In addition, a C-peptide variant can show at least one in insertion compared to wild type C-peptide, and preferably up to 50, more preferably up to 40 and most preferably up to 30 insertions. The insertions are preferably at the C- and/or N-terminal end.

Preferred ligands according to the present invention in particular comprise a C-peptide sequence variant, wherein one of the amino acids no. 12 (L), no. 21 (L), no. 22 (Q) or no. 23 (P) is substituted by another amino acid. Particularly preferred are substitutions of L(12) to C or P, in particular to C, substitution of P(23) to A, V or S, in particular to A, substitution of L(21) to T, I, F, C, Q, V or S, in particular to I or substitution of Q(22) to S, A, N or M, in particular to A or to S.

These preferred C-peptide sequence variants can show, besides substitutions of one of the amino acids nos. 12 (L), no. 21 (L), no. 22 (Q) or no. 23 (P) one or more further amino acid substitutions and/or deletions. Preferably, the C-peptide variant shows up to 25 further substitutions and/or deletions.

The ligands further can be truncated compared to the wild type C-peptide on the N-terminal, the C-terminal or on the N and C-terminal by 1 to 25, in particular by 1 to 20, preferably 2 to 9, more preferably 3 to 6 amino acids. In one embodiment, a truncated peptide is used as ligand consisting of amino acids 21-31 of SEQ ID NO: 1. Preferred ligands show amino acids 10 to 25 of SEQ ID NO: 1. In one embodiment, the ligand comprises the stretch of amino acids between positions 10 to 25 of SEQ ID NO:1, wherein however one to four or one to two of the amino acids between positions 10 to 25 of SEQ ID NO:1 are substituted, in particular the amino acid no. 21 (L), no. 22 (Q) and/or no. 23 (P). In one embodiment, the ligand comprises the stretch of amino acids between positions 10 to 25 of SEQ ID NO:1, wherein however one of the amino acids between positions 10 to 25 of SEQ ID NO:1 is substituted, in particular the amino acid no. 21 (L), no. 22 (Q) or no. 23 (P).

It is particularly preferred that the ligand is a variant C-peptide selected from

Mut2: AA10-25; P(23)to A
(SEQ ID NO: 2)
N-VELGGGPGAGSLQALA-C
Mut3: AA10-25; L(21)to I
(SEQ ID NO: 3)
N-VELGGGPGAGSIQPLA-C
Mut5: AA10-25; Q(22)to S
(SEQ ID NO: 4)
N-VELGGGPGAGSLSPLA-C
Mut6: AA10-25; Q(22)to A
(SEQ ID NO: 5)
N-VELGGGPGAGSLAPLA-C
Mut18: AA8-17; L(12)to C
(SEQ ID NO: 6)
N-GQVECGGGPG-C

Further preferred ligands are selected from:

Mut6-Cys: AA10-25; Q(22) to A and including a cysteine linker

(SEQ ID NO: 7)
N-CGSGSGSGS-VELGGGPGAGSLAPLA-C
Mut21: AA21-31
(SEQ ID NO: 8)
N-LQPLALEGSLQ-C
Mut22: AA21-31; Q(22) to A
(SEQ ID NO: 9)
N-LAPLALEGSLQ-C

In further preferred embodiments the ligand comprises a cysteine linker or a biotin group.

Further suitable C-peptide variants showing a reduced binding to the anti-C-peptide antibody show one of the following substitutions:
A(25) substituted by Y, Q, G, W, N, F, C, I, H, P, R, M or V
L(24) substituted by I, V, T, Y, Q, M or F
P(23) substituted by A, S or V
Q(22) substituted by N, M, S or A
L(21) substituted by F, T, I, Q, V, S, C
S(20) substituted by Q, N, T, V, L, I, A, Y or M
G(19) substituted by S, N, L, A, P or Q
A(18) substituted by M, I, V, T, R, S, Q, G or K
G(17) substituted by T, Y, H, M, N, I, K, S or R
P(16) substituted by L, R, Y, M, N, Q, I, H, K, T, S or C
G(15) substituted by Y, H, T, S, Q, K, P, M or R
G(14) substituted by Y, A, P, V, H, S, R, L, C, M or N
G(13) substituted by Y, R, T, I, S, P or N
L(12) substituted by C, Y, S, P, Q, M, N, T, W, R or I
E(11) substituted by Y, W, Q, N or P
V(10) substituted by Y, W, Q, N, I, L, E, M or P

A further subject matter of the present invention is the provision of ligands as described herein, in particular the described C-peptide variants.

In a particularly preferred embodiment of the present invention the displacement assay comprises the steps
a) providing a solid phase,
b) immobilizing an anti-C-peptide antibody on the solid phase,
c) contacting the anti-C-peptide antibody with a ligand to provide an antibody-ligand-complex,
d) contacting the antibody-ligand-complex with a sample to be analyzed,
e) displacement of the ligand from the antibody-ligand-complex by C-peptide analyte and
f) determining the amount of displaced ligand.

In this embodiment the anti-C-peptide antibody is immobilized on a solid phase. Suitable solid phases include particles, such as beads or test strips. Preferably, a test strip is provided. The surface of the solid phase is preferably made of a polymer such as polystyrene, polyethylene, cellulose or the like or of a metal, in particular a noble metal such as Au, or a conductive carbon structure such as graphene or carbon nanotubes.

The antibody can be directly immobilized on the solid surface. However, it is also possible in some embodiments to couple the antibody to the solid surface via a linker or via a high affinity binding pair such as biotin/streptavidin.

The anti-C-peptide antibody immobilized onto the solid phase is then contacted with a ligand, as herein described to provide an antibody-ligand-complex. In this embodiment the antibody-ligand-complex is immobilized on the solid phase via the antibody.

The ligand may contain a label, such as an optical label for giving a fluorescence signal, a polarization signal, a FRET signal or similar, or the label may be an enzyme. However, it is also possible to use ligands having no label and performing a label-free detection, such as impedance measurement, weight measurement, or electron transfer measurement.

In a next step the immobilized antibody-ligand-complex is contacted with a sample to be analyzed. Due to the high affinity of C-peptide analyte and in particular wild type C-peptide compared to the ligand, the ligand is displaced from the antibody-ligand-complex by the C-peptide analyte and in particular wild type C-peptide and the ligand is released into the sample. Based on the displacement of ligand by C-peptide analyte and/or on release of ligand from the antibody-ligand-complex the amount of displaced ligand can be determined. Based thereon the amount of C-peptide analyte within the sample can be determined. Preferably, the amount of C-peptide analyte is quantified by calibration of the assay using samples having known C-peptide analyte concentrations.

In case a labeled ligand is employed the amount of released ligand and thus the amount of C-peptide analyte and in particular wild type C-peptide within a sample can be determined by measuring the label.

Alternatively, the amount of released ligand can be determined by label-free detection, such as weight measurement or impedance measurement of constructs attached to the solid phase.

In a particularly preferred embodiment a test strip is used for performing the displacement assay. Such a test strip comprises a sample application port, in particular for applying blood, serum or urine. Next an optional net or fleece is provided for removal of blood cells. Further, such a test strip may contain optionally a membrane for removal of other high molecular weight compounds. Further down the flow path a test field is provided which contains the immobilized antibody-ligand-complex. Released free ligand is then determined at a detector further down the flow path.

In a further particularly preferred embodiment of the present invention the displacement assay comprises the steps
a) providing a solid phase,
b) immobilizing a ligand on the solid phase,
c) contacting the ligand with an anti-C-peptide antibody to provide an antibody-ligand-complex,
d) contacting the antibody-ligand-complex with a sample to be analyzed,
e) displacement of the ligand from the antibody-ligand-complex by C-peptide analyte and
f) determining the amount of displaced ligand.

In this embodiment as solid phase preferably a test strip is used comprising a polymer, such as polyethylene or polystyrene or a metal, in particular an Au-surface or a conductive carbon structure such as graphene or carbon nanotubes. On the surface of the solid phase a ligand is immobilized. Immobilizing of the ligand can e.g. be performed via a self-assembling monolayer or via a linker. In a particularly preferred embodiment using a solid phase having an Au-surface, first a self-assembled monolayer (SAM) is applied. Such a SAM prevents unspecific adsorption of peptides or proteins being within the sample liquid onto the surface. Part of the self-assembled monolayer molecules is coupled to a high affinity binding partner, such as biotin which in turn is coupled to its binding partner streptavidin. Onto this modified surface biotin-labeled ligands can be immobilized. By contacting the immobilized ligand with an anti-C-peptide antibody an immobilized antibody-ligand-complex is generated. In this embodiment the antibody-ligand-complex is immobilized on the solid phase via the ligand.

In a next step the immobilized antibody-ligand-complex is contacted with a sample to be analyzed. C-peptide analyte and in particular wild type C-peptide contained in the sample displaces the ligand from the antibody-ligand-complex. Thereby an antibody-C-peptide analyte-complex is released while immobilized ligand remains at the solid phase. Detection of the amount of C-peptide analyte and in particular wild type C-peptide contained in a sample can be performed using several techniques. E.g. the anti-C-peptide antibody employed can be labeled and the amount of released antibody-C-peptide analyte-complex can be measured using this label. Alternatively, a label-free detection method is employed, such as impedance measurement, weight measurement, or electron transfer measurement.

However, it is also possible to measure the amount of the construct bound to the surface, e.g. by QCM (Quartz Crystal-microbalance). In this technique a frequency shift shows increase or decrease of the weight of a construct attached to the solid phase surface.

Thus the amount of displaced ligand can be measured directly by measuring the amount of free ligand or indirectly by measuring the amount of released antibody.

The inventive immunoassay for measuring C-peptide using a ligand displacement method is in particular suitable in the individualized monitoring and treatment of subjects having or being suspected to have prediabetes, Type 1 diabetes or Type 2 diabetes. The immunoassay is in particular used for diagnosis or differential diagnosis with regard to diabetes mellitus.

The invention further relates to a C-peptide variant as described herein. In particular, the invention relates to a C-peptide variant, wherein one of the amino acids no. 12 (L), no. 21 (L), no. 22 (Q) or no. 23 (P) of SEQ ID NO: 1 is substituted.

A C-peptide variant according to the present invention preferably comprises a stretch of at least 6, preferably at least 7, more preferably at least 8, in particular at least 9 and most preferably at least 10 consecutive amino acids of SEQ ID NO: 1, in which stretch of amino acid sequence 1, 2 or 3, preferably 1 or 2 and most preferably 1 amino acid may be substituted, and up to 31, preferably up to 25 and more preferably up to 20 consecutive amino acids of SEQ ID NO: 1. In one embodiment, a C-peptide variant according to the present invention comprises a stretch of at least 15 consecutive amino acids of SEQ ID NO: 1, in which stretch of amino acid sequence however 1, 2 or 3, preferably 1 or 2 and most preferably 1 amino acid may be substituted. Particularly preferred is a C-peptide variant, wherein one of the amino acids no. 12 (L), no. 21 (L), no. 22 (Q) or no. 23 (P) of SEQ ID NO: 1 is substituted and which is further truncated at the N- and/or C-terminal, in particular truncated by up to 25 amino acids, preferably up to 20 amino acids. Particularly preferred are C-peptide variants, wherein the amino acid no. 21 (L) is substituted by T, I, F, C, Q, V or S, in particular by I, and/or wherein the amino acid no. 22 (Q) is substituted by S, A, N or M, in particular by A, and/or wherein the amino acid no. 23 (P) is substituted by A, V or S, in particular by A or S and/or wherein the amino acid no. 12 (L) is substituted by C or P, in particular by C.

The invention further relates to a test kit for performing a displacement assay as described herein, which test kit comprises
a) optionally a solid phase,
b) an anti-C-peptide antibody, and
c) a ligand having a lower affinity to the anti-C-peptide antibody and wild type C-peptide.

Preferred embodiments of the constituents of the test kit are solid phases, anti-C-peptide antibodies and ligands as described herein.

The invention is further illustrated by the enclosed Figures and the following Examples.

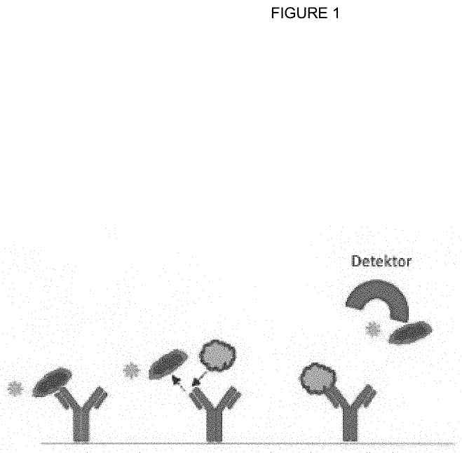

FIG. 1 shows a schematic scheme of a displacement assay, wherein an antibody is immobilized to a solid phase to which immobilized antibody a labeled ligand is coupled. This labeled ligand is displaced by the analyte resulting in release of labeled ligand, which can be measured by a detector.

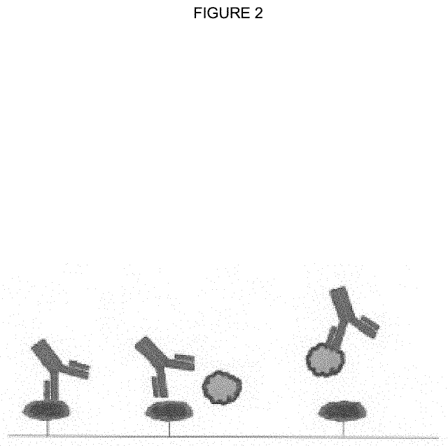

FIG. 2 shows a schematic scheme of a displacement assay, wherein a ligand is immobilized to a solid phase to which ligand an antibody is coupled. By adding a sample comprising analyte, the ligand is displaced from the antibody-ligand-complex. The ligand remains immobilized on the solid phase and an analyte-antibody-complex is released from the surface.

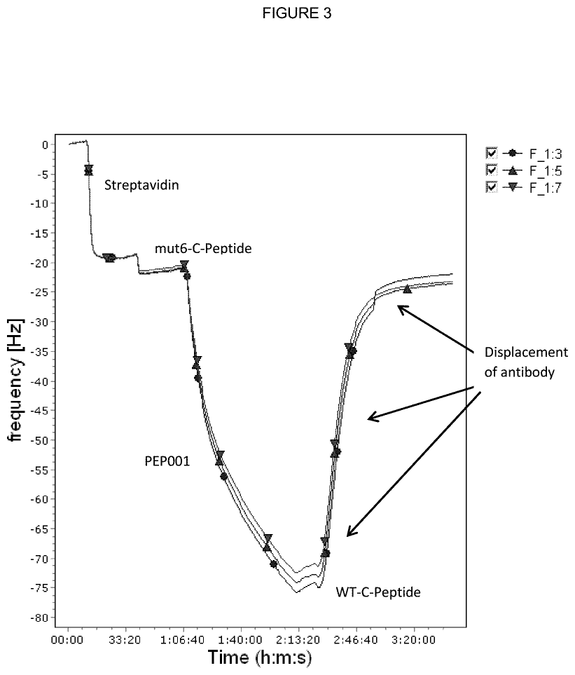

FIG. 3 shows QCM-data (Quartz Crystal Microbalance data) of a stepwise performance of a displacement immunoassay. A negative frequency shift shows increase of mass attached to the surface (in this case an Au-surface). Ligand used: mut6-C-peptide.

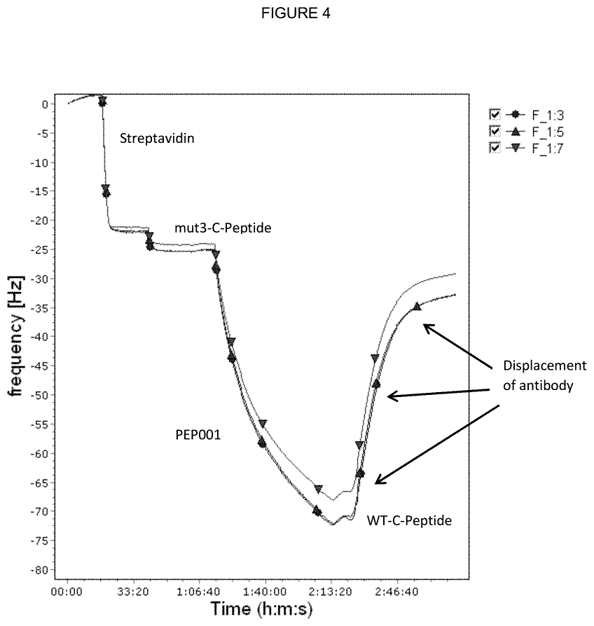

FIG. 4 shows a further QCM-measurement of an displacement immunoassay. Ligand used: mut3-C-peptide.

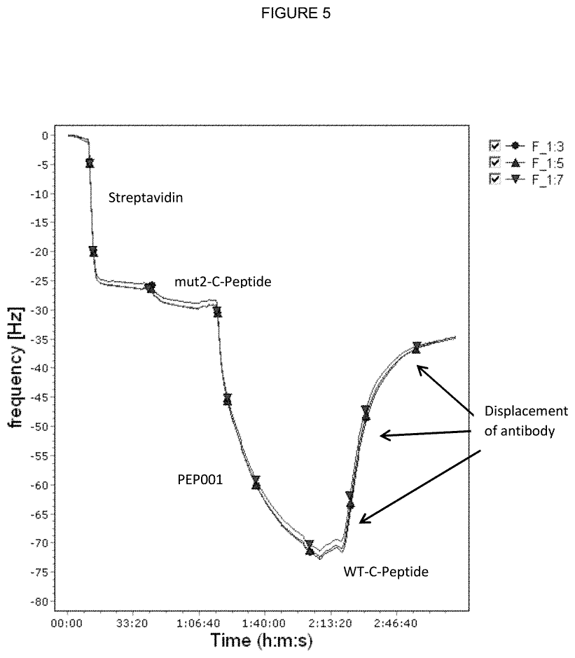

FIG. 5 shows a further QCM-measurement of an displacement immunoassay. Ligand used: mut2-C-peptide.

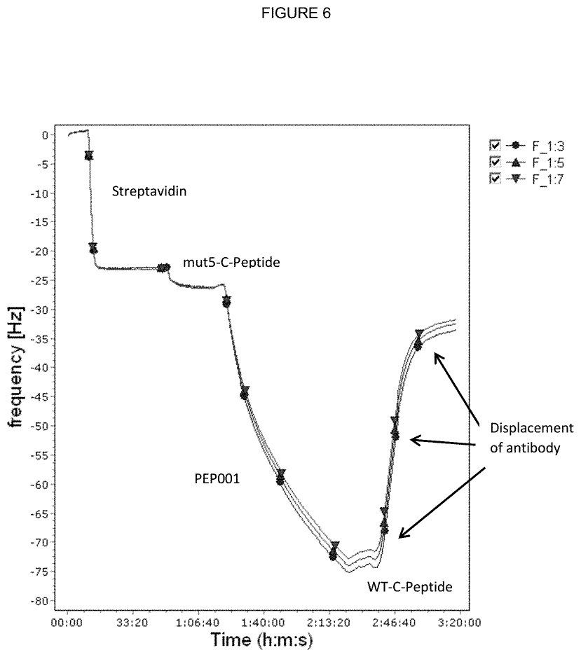

FIG. 6 shows a further QCM-measurement of an displacement immunoassay. Ligand used: mut5-C-peptide.

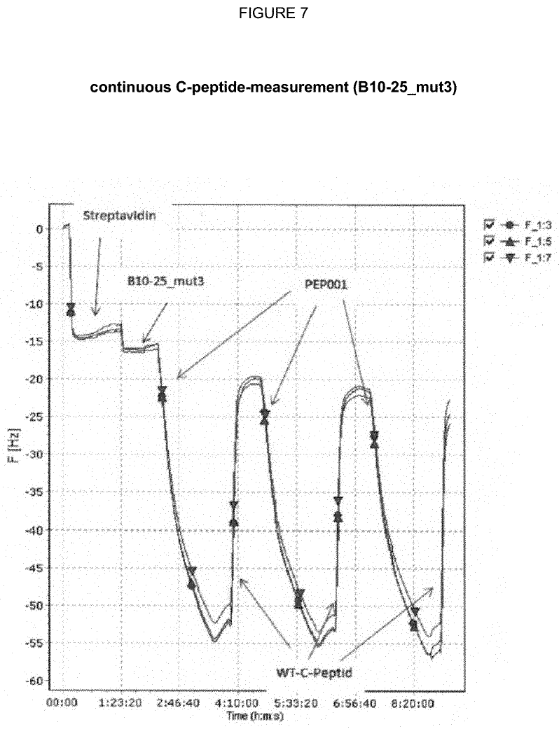

FIG. 7 shows a continuous C-peptide measurement.

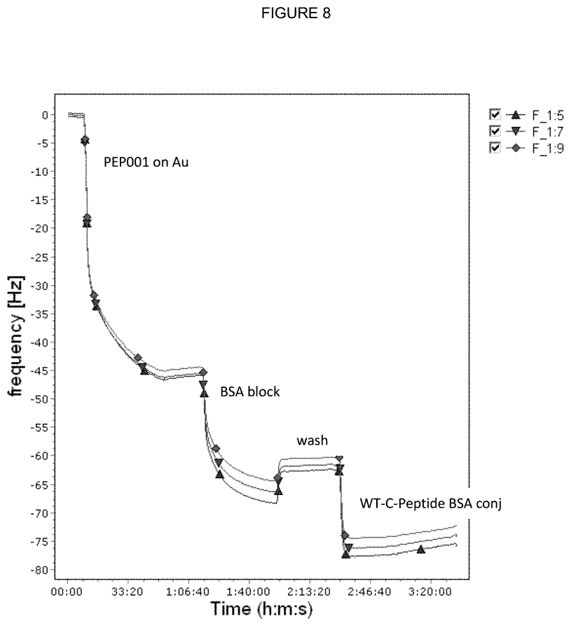

FIG. 8 shows a QCM-measurement, wherein a PEP001 antibody was immobilized on an Au-surface.

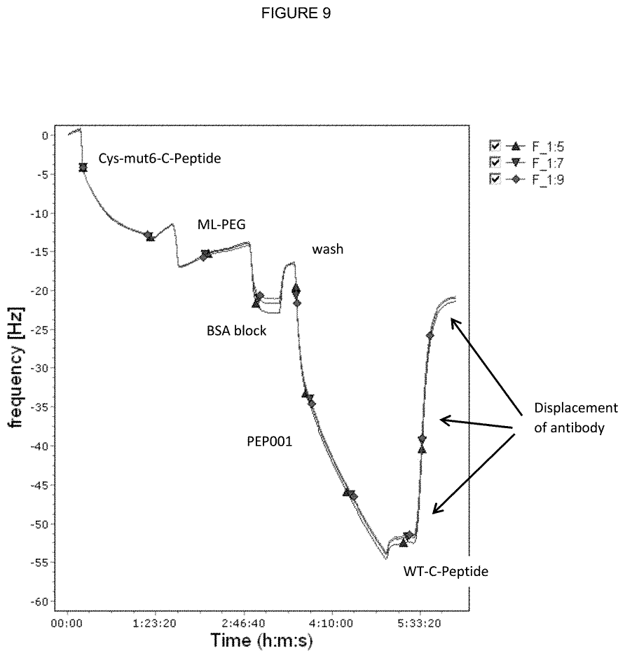

FIG. 9 shows a displacement immunoassay, wherein a ligand is coupled to the solid surface using a linker.

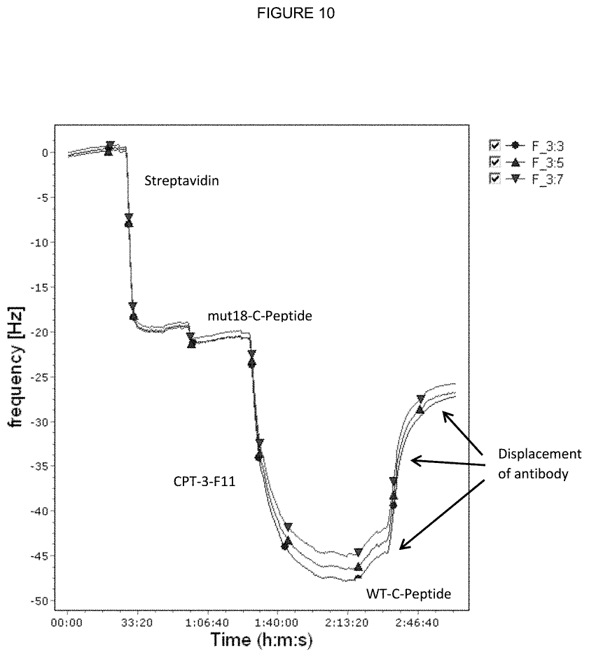

FIG. 10 shows a further QCM-measurement of an displacement immunoassay. Antibody used: CPT-3-F11, ligand used: mut18-C-peptide.

### EXAMPLES

### Example 1: Displacement Immunoassay for Detecting C-Peptide Using Mut6-C-Peptide

An Au-substrate is covered with a self-assembled monolayer (SAM). This self-assembled monolayer protects the surface from unspecific adsorption of peptides or proteins. Part of the SAM molecules is coupled with biotin, which is exposed on the surface. To this surface the biotin binding protein streptavidin is applied and bound by the biotin moieties. Since streptavidin has a plurality of binding sides for biotin, a ligand labeled with biotin can be bound to the streptavidin. Finally, anti-C-peptide antibody PEP001 is added, which binds to the ligand forming an immobilized antibody-ligand-complex, which is schematically depicted in FIG. 2. The stepwise build-up of an immobilized antibody-ligand-complex on the Au-surface can be monitored using a Quartz Crystal Microbalance (QCM). A negative frequency shift shows increase of mass on the Au-surface. Referring to FIG. 3, attachment of streptavidin and Mut6-C-peptide results in a frequency shift of about −20 Hz. The binding of antibody PEP001 to the ligand Mut6-C-peptide results in a frequency shift of about >−50 Hz.

Addition of a sample containing C-peptide results in displacement of the ligand by wild type C-peptide and release of a C-peptide-antibody-complex as schematically shown in FIG. 2. In FIG. 3 release of the antibody-C-peptide-complex results in an observable positive frequency shift.

A quantitative determination of the amount of C-peptide within a sample can be achieved by calibrating the assay using solutions having known C-peptide concentrations. The amount of frequency shift observed for a sample indicates the quantitative amount of C-peptide being present within that sample.

### Example 2: Displacement Immunoassay for C-Peptide Using Mut3-C-Peptide, Mut2-C-Peptide or Mut5-C-Peptide

An immunoassay as described in Example 1 was provided except that Mut3-C-peptide, Mut2-C-peptide or Mut5-C-peptide were used as ligand.

As can be seen in FIG. 4, the stepwise build-up of a streptavidin/Mut3-C-peptide/PEP001 assembly results in a mass build-up on the surface which can be seen as negative frequency shift in the QCM-data. Addition of a sample containing WT-C-peptide (shortly after 2 hours and 15 minutes) results in displacement of the ligand Mut3-C-peptide by WT-C-peptide and in a considerable frequency increase. This frequency increase can directly be correlated to the amount of WT-C-peptide in the sample. FIG. 5 and FIG. 6 show similar results for Mut2-C-peptide and Mut5-C-peptide respectively.

### Example 3: Continuous Immunoassay for Detection of C-Peptide

As described in Example 1, a SAM/biotin/streptavidin/biotin-ligand/antibody-complex is immobilized on an Au-surface. In this Example the ligand was C-peptide mutant B10-25_Mut3, the antibody was PEP001. The stepwise build-up monitored using QCM is shown in FIG. 7. Adding a sample containing 500 ng/ml WT-C-peptide results in release of antibody-WT-C-peptide-complex, which is observed in the QCM-data as positive frequency shift (addition of WT-C-peptide at about 4 hours).

It is then possible to regenerate the assay by again adding antibody PEP001 forming ligand/antibody-complex immobilized to the surface. Thus, it is possible to use the assay for a second and further measurement. Exemplary in FIG. 7, three consecutive measurements are shown.

### Example 4: Displacement Immunoassay for C-Peptide Using an Immobilized Antibody

Antibody PEP001 was directly immobilized to an Au-surface as shown in FIG. 1. Unspecific protein binding sides were saturated with BSA (Bovine Serum Albumin). Then a WT-C-peptide covalently coupled with BSA was added to reveal build-up of an immobilized antibody-C-peptide-complex. The results are shown in FIG. 8. Due to the weight of BSA (70 kDa) the binding of C-peptide to the immobilized can be made visible.

### Example 5: Displacement Immunoassay Using a Linker to Immobilize a Ligand to a Surface

Immobilization of a ligand to a surface can also be performed using a linker (instead of a self-assembling monolayer). A suitable linker is for example a cysteine linker. In this configuration immobilization of an antibody via an antibody-ligand-complex to the surface can be detected by measuring impedance. A ligand having the sequence N-CGSGSGSGSVELGGGPGAGSLAPLA-C(SEQ ID NO:7) was directly immobilized on an Au-surface. Additionally, 6-ferrocenyl-1-hexanethiol was bound to the Au surface for enhanced impedance measurement. Unspecific protein binding to the surface was avoided by incubation with a polyethylene glycol (ML-PEG) and bovine serum albumin (BSA). The QCM-data for this assay are shown in FIG. 9. Addition of PEP001 antibody results in a considerable negative shift of the frequency and addition of WT-C-peptide results in release of the bound antibody.

### Example 6: Displacement Immunoassay for C-Peptide Using Mut18-C-Peptide

An immunoassay as described in Example 1 was provided except that Mut18-C-peptide was used as ligand.

As can be seen in FIG. 10, the stepwise build-up of a streptavidin/Mut18-C-peptide assembly results in a mass build-up on the surface which can be seen as negative frequency shift in the QCM-data. Addition of the anti-C-peptide antibody CPT-3-F11 resulted in a frequency shift of almost −30 Hz. Addition of a sample containing WT-C-peptide (shortly after 2 hours and 15 minutes) results in displacement of the ligand Mut18-C-peptide by WT-C-peptide and in a considerable frequency increase. This frequency increase can directly be correlated to the amount of WT-C-peptide in the sample.

1. A method for detecting C-peptide comprising:
i) providing an antibody-ligand-complex, wherein the antibody is an antibody directed against C-peptide and wherein the ligand has a lower affinity to the anti-C-peptide antibody than wild type C-peptide;
ii) contacting the antibody-ligand-complex with a sample to be analyzed; and
iii) displacing the ligand from the antibody-ligand-complex by C-peptide analyte, thereby determining the presence or concentration of C-peptide analyte within the sample.

2. The method according to claim 1, wherein the ligand is a C-peptide variant, in particular a C-peptide variant wherein one of the amino acids no. 12 (L), no. 21 (L), no. 22 (Q) and/or no. 23 (P) is substituted and comprising optionally up to 25 further substitutions and/or deletions.

3. The method according to claim 1, wherein the ligand is a C-peptide variant, wherein the amino acid no. 21 (L) is substituted by T, I, F, C, Q, V or S, in particular by I, and/or wherein the amino acid no. 22 (Q) is substituted by S, A, N or M, in particular by A or S, and/or wherein the amino acid no. 23 (P) is substituted by A, V or S, in particular by A, and/or wherein the amino acid no. 12 (L) is substituted by C or P, in particular by C.

4. The method according to claim 1, wherein the ligand is a C-peptide variant, wherein 1 to 20 amino acids are deleted at the C-terminus and/or at the N-terminus.

5. The method according to claim 1, wherein the ligand is a variant C-peptide selected from
Mut2: AA10-25; P(23)to A
(SEQ ID NO: 2)
N-VELGGGPGAGSLQALA-C
Mut3: AA10-25; L(21)to I
(SEQ ID NO: 3)
N-VELGGGPGAGSIQPLA-C
Mut5: AA10-25; Q(22)to S
(SEQ ID NO: 4)
N-VELGGGPGAGSLSPLA-C
Mut6: AA10-25; Q(22)to A
(SEQ ID NO: 5)
N-VELGGGPGAGSLAPLA-C
Mut18: AA8-17; L(12)to C
(SEQ ID NO: 6)
N-GQVECGGGPG-C

6. The method according to claim 1, wherein the ligand comprises a label and/or a linker.

7. The method according to claim 1, wherein the ligand comprises a label selected from biotin, optical labels, electrochemical labels, enzyme labels and/or mass label.

8. The method according to claim 1 being a displacement assay comprising the steps
a) providing a solid phase,
b) immobilizing an antibody directed against C-peptide on the solid phase,
c) contacting the antibody with a ligand to provide an antibody-ligand-complex,
d) contacting the antibody-ligand-complex with a sample to be analyzed,
e) displacement of the ligand from the antibody-ligand-complex by C-peptide analyte, and
f) determining the amount of displaced ligand.

9. The method according to claim 1, being a displacement assay comprising the steps
a) providing a solid phase,
b) immobilizing a ligand on the solid phase,
c) contacting the ligand with an anti-C-peptide antibody to provide an antibody-ligand-complex,
d) contacting the antibody-ligand-complex with a sample to be analyzed,
e) displacement of the ligand from the antibody-ligand-complex by C-peptide analyte and
f) determining the amount of displaced ligand.

10. A test kit for performing the method of claim 1 comprising
a) optionally a solid phase,
b) an anti-C-peptide antibody, and
c) a ligand having a lower affinity to the anti-C-peptide antibody then wild type C-peptide.

11. The test kit according to claim 10, wherein the anti-C-peptide antibody and/or the ligand comprise a label.

12. The test kit according to claim 10 comprising an antibody-ligand-complex immobilized to a solid phase.

13. A C-peptide variant, wherein one of the amino acids no. 12 (L), no. 21 (L), no. 22 (Q) or no. 23 (P) of SEQ ID NO: 1 is substituted.

14. The C-peptide variant of claim 13, wherein the amino acid no. 21 (L) is substituted by T, I, F, C, Q V or S, in particular by I, and/or wherein the amino acid no. 22 (Q) is substituted by S, A, N or M, in particular by A or S, and/or wherein the amino acid no. 23 (P) is substituted by A, V or S, in particular by A, and/or wherein the amino acid no. 12 (L) is substituted by C or P, in particular by C.

15. The C-peptide variant of claim 13, wherein the amino acid no. 21 (L) is substituted by T, F, C, V or S, and/or wherein the amino acid no. 22 (Q) is substituted by S, A, N or M, in particular by A or S, and/or wherein the amino acid no. 23 (P) is substituted by A, V or S, in particular by A, and/or wherein the amino acid no. 12 (L) is substituted by C or P, in particular by C.


SEQUENCE LISTING (9 sequences)

SEQ ID NO: 1 - protein, 31 aa, Homo sapiens
EAEDLQVGQVELGGGPGAGSLQPLALEGSLQ

SEQ ID NO: 2 - protein, 16 aa, Artificial Sequence
  variant C-peptide Mut2
VELGGGPGAGSLQALA

SEQ ID NO: 3 - protein, 16 aa, Artificial Sequence
  variant C-peptide Mut3
VELGGGPGAGSIQPLA

SEQ ID NO: 4 - protein, 16 aa, Artificial Sequence
  variant C-peptide Mut5
VELGGGPGAGSLSPLA

SEQ ID NO: 5 - protein, 16 aa, Artificial Sequence
  variant C-peptide Mut6
VELGGGPGAGSLAPLA

SEQ ID NO: 6 - protein, 10 aa, Artificial Sequence
  variant C-peptide Mut18
GQVECGGGPG

SEQ ID NO: 7 - protein, 25 aa, Artificial Sequence
  variant C-peptide Mut6-Cys
CGSGSGSGSVELGGGPGAGSLAPLA

SEQ ID NO: 8 - protein, 11 aa, Artificial Sequence
  variant C-peptide Mut21
LQPLALEGSLQ

SEQ ID NO: 9 - protein, 11 aa, Artificial Sequence
  variant C-peptide Mut22
LAPLALEGSLQ